(19) United States
(12) Patent Application Publication
(10) Pub. No.: US 2025/0042869 A1
(43) Pub. Date: Feb. 6, 2025

(54) HETEROGENEOUS STANNOUS OXIDE CATALYSTS FOR THE LOW COLOR ESTERIFICATION OF FURAN-2,5-DICARBOXYLIC ACID

(71) Applicant: Archer-Daniels-Midland Company, Decatur, IL (US)

(72) Inventors: Chi-Cheng MA, Champaign, IL (US); Emily Nehrkorn, Decatur, IL (US); Kenneth F. Stensrud, Decatur, IL (US); Erik Hagberg, Decatur, IL (US); William C. Hoffman, Decatur, IL (US)

(21) Appl. No.: 18/577,187

(22) PCT Filed: Jul. 5, 2022
(86) PCT No.: PCT/US2022/036154

§ 371 Date: Jan. 5, 2024

Related U.S. Application Data
(60) Provisional application No. 63/219,127, filed on Jul. 7, 2021.

(51) Int. Cl. C07D 307/68; B01J 23/14; B01J 35/00

(52) U.S. Cl. CPC C07D 307/68; B01J 23/14; B01J 35/19

(57) ABSTRACT

An improved process and catalyst are provided for making an FDCA diester monomer product with improved productivity compared to an autocatalyzed esterification but with comparable or at least not greatly diminished color properties compared to the FDCA diester monomer product that would be produced autocatalytically, wherein a heterogeneous tin (II) catalyst is employed to make an esterification product comprising a diester of FDCA with an alcohol, the catalyst being in either a bulk, unsupported form or in the form of a supported tin (II) catalyst, in particular, using a hygroscopic support such as a gamma alumina, a zeolite or a silica, or using a carbon support.

### FIELD OF THE INVENTION

The present disclosure pertains generally to the esterification of sugar-derived furan-2,5-dicarboxylic acid (FDCA), and more particularly, to catalysts used in these esterifications.

### BACKGROUND

Petroleum traditionally has been a primary source of raw material for making organic monomer precursors for common polymeric materials. However, with concerns about climate change and carbon dioxide emissions from fossil fuel sources, researchers have turned to biologically based and thus renewable resources for the development of plausible surrogates for these traditionally petroleum-derived monomers.

Carbohydrates, sometimes simply termed sugars, are a diverse class of organic materials providing extended, carbon-chained building blocks from which such biobased surrogates might be made. For over 150 years, scientists have explored various chemical means for adapting the properties of sugars to an array of applications, including for making polymers therefrom. Dehydrative cyclization is a common transformation that sugars can undergo, particularly at elevated temperatures and in the presence of a catalyst, producing furan-based substances. For example, the common sugar, fructose, readily cyclizes at low pH to produce a versatile precursor, 5-hydroxymethyl-2-furfural (hereafter, HMF). This process is illustrated in Scheme A.

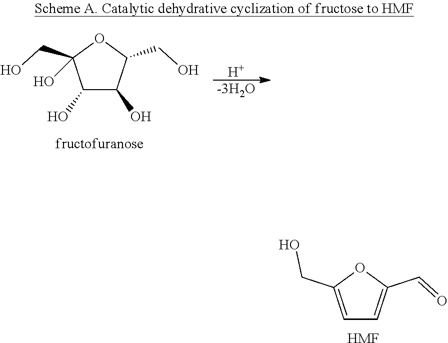

By virtue of its unique functionality, HMF can, in turn, be modified into other interesting molecular entities, such as furan-2,5-dimethanol (FDM), 2,5-bishydroxymethyl tetrahydrofuran (bHMTHF), diformylfuran (DFF), and 2,5-furandicarboxylic acid (hereafter, FDCA).

FDCA and its ester derivatives, especially its diester derivative with methanol (2,5-furandicarboxylic acid, dimethyl ester (FDME)), have recently attracted a great deal of interest for the production of poly(alkylene furan dicarboxylate) polymers that can substitute for their petroleum derived analogs, namely poly(alkylene terephthalate) polymers, such as polyethylene terephthalate (PET). Prominent examples of poly(alkylene furan dicarboxylate) polymers are poly(ethylene furan dicarboxylate), or PEF, and poly(trimethylene furan dicarboxylate), or PTF, in which the different polymer backbones of these polyesters are respectively obtained by reaction of FDCA or of an ester derivative of FDCA, such as FDME, with the different co-monomers of ethylene glycol and 1,3-propane diol. In addition to the desirable genesis in carbohydrate rather than petroleum-based feedstocks for the FDCA or FDCA ester monomer (and recognizing as well that a wholly biobased ethylene glycol (1,2-ethanediol) with which the FDCA or FDCA ester monomer is reacted is currently being produced and used in combination with purified terephthalic acid to make a partly biobased PET), the bio-plastic PEF has been found to provide superior properties in a number of respects, relative to the petroleum derived analog PET, particularly in the area of packaging. For example, blends of PEF and PET can provide improved barrier properties with respect to CO₂ and O₂, prolonging shelf life over pure PET and providing an acceptable container for products such as beer which are susceptible to oxidative degradation. Other packaging applications of PEF include films used to manufacture pouches, wrappers, and heat shrink materials having high mechanical strength and recyclability.

In general, both FDCA and its esters such as FDME thus show substantial promise as plausible surrogates for terephthalic acid and its diesters, respectively, in the production of polyamides, polyurethanes, and polyesters having diverse applications as plastics, fibers, coatings, adhesives, personal care products, and lubricants.

One important consideration in realizing commercially acceptable, renewable resource-based polymers for many of these applications is the color performance of the polymers. Color, or more appropriately the absence of color, is an important attribute for the polymers to be made from FDCA and/or an ester of FDCA rather than from their corresponding non-renewable analogs in applications such food packaging and particularly beverage bottle manufacturing, in which a lack of transparency or possible yellowness in the plastic are readily perceived and may be equated to an inferior product.

U.S. Pat. No. 9,567,431 prescribes one approach to providing suitably low color polyesters from these sugar-derived materials, relating a two-step process wherein first a prepolymer is made having a 2,5-furandicarboxylate moiety within the polymer backbone. This intermediate product is described as preferably an ester composed of two diol monomers and one diacid monomer, wherein at least part of the diacid monomers comprises 2,5-FDCA. This first, prepolymerization step is then followed by a melt-polymerization of the prepolymers under suitable polymerization conditions.

The US'431 reference indicates that it is “essential” that the first step is a transesterification step, “catalyzed by a specific transesterification catalyst at a temperature preferably in the range of from about 150 to about 220° C., more preferably in the range of from about 180 to about 200° C. and carried out until the starting ester content is reduced until it reaches the range of about 3 mol % to about 1 mol %”. This specific transesterification catalyst may then be removed to avoid interaction in the second step of polycondensation but is indicated as typically included in the second step without any purification of the product from the prepolymerization step. In particular, tin(IV) based catalysts, preferably organotin (IV) based catalysts, more preferably alkyltin (IV) salts including monoalkyltin (IV) salts, dialkyl and trialkyltin (IV) salts and mixtures thereof, are indicated, and are described as superior to tin (II) based catalysts such as tin (II) octoate. Preferred transesterification catalysts are selected from one or more of, butyltin (IV) tris(octoate), dibutyltin (IV) di(octoate), dibutyltin (IV) diacetate, dibutyltin (IV) laureate, bis(dibutylchlorotin(IV)) oxide, dibutyltin dichloride, tributyltin (IV) benzoate and dibutyltin oxide.

For the second, polycondensation step, the US'431 reference indicates that it is “essential” that this second step is “catalyzed by a specific polycondensation catalyst and that the reaction is carried out at mild melt conditions”, with examples of the “specific polycondensation” catalysts including tin (II) salts such as tin (II) oxide, tin (II) dioctoate, butyltin (II) octoate, or tin (II) oxalate. Preferred catalysts according to the US'431 reference are those tin (II) salts which are obtained by the reduction of the tin (IV) catalyst, e.g., alkyltin (IV), dialkyltin (IV), or trialkyltin (IV) salts, used as the transesterification catalyst with a reducing compound, for example, organophosphorus compounds of trivalent phosphorus, in particular a monoalkyl or dialkyl phosphinate, a phosphonite or a phosphite.

In general, however, for FDCA- and FDCA ester-based polymers to acquire a level of commercial acceptance as acceptable alternatives to those based on terephthalic acid and terephthalate esters, there remains a need to more fundamentally address undesirable color formation associated with the use of FDCA and/or FDCA esters per se.

The commercial realization of FDCA as a renewable alternative to terephthalic acid has also been hindered by the more challenging physical attributes of FDCA as compared to terephthalic acid (e.g., its limited solubility in many common organic solvents and its extremely high melting point (>300° C.)), in respect both of its synthesis and subsequent use in such promising applications. The high melting point of FDCA, for example, poses difficulties for employing FDCA in conventional melt polymerization processing methods. Simple chemical modifications, such as esterification, have long been used in relation to other similarly challenging materials to overcome the barriers that arise from a desired product's physical properties. Consequently, it has been considered that the esterification of FDCA with methanol, for example, to make its methyl esters, and especially to make its dimethyl ester (dimethyl 2,5-furandicarboxylate (hereafter FDME)) would provide an appreciably more manageable and user-friendly prospective monomer, having a much-reduced melting point (112° C.) and boiling point (140-145° C. (10 torr)), and further having improved solubility compared to FDCA in a number of commonly used organic solvents.

Unfortunately, the esterification of FDCA is not without its own challenges. Over the years, esterification by autocatalysis has been demonstrated in a large number of publications. The inherent inefficiency of this process, however, arises from the need to use high temperatures and long, protracted reaction times (and typically high molar excesses of the alcohol as well) to attain desired yields of the favored corresponding FDCA diester product. These factors would contribute significantly to the foreseeable cost of manufacturing diesters of FDCA on an industrial or commercial scale. Brønsted acid catalysis greatly improves FDCA conversions and ester yields, but also readily drives alcohol condensation to undesired, low molecular weight ethers. The generation of byproducts represents a yield loss and complicates downstream processing. Lewis acid catalysts have been proposed as well, but often suffer from limited activity (lability) and from a propensity to generate undesired Brønsted acids when in an aqueous matrix.

WO 2018/093413 is of interest in comparing the relative performance of tin (II) and tin (IV) salts as homogeneous catalysts for the direct esterification of FDCA to especially its dimethyl ester, FDME, finding, in common with the US'431 reference in a different, transesterification/prepolymerization context, that the homogeneous tin (IV) salts were to be preferred over the homogeneous tin (II) salts. While very good yields of FDME were obtained after a relatively brief reaction time and under relatively mild conditions, the use of homogeneous tin salts as esterification catalysts requires an effective means for recovery of the tin and also produces an FDME product that is more highly colored than that produced by autocatalysis.

Further improvements in the esterification of FDCA are consequently also still needed.

### SUMMARY OF THE INVENTION

The present invention from one perspective addresses the need for a low color FDCA diester monomer product, by providing a heterogeneous tin (II) catalyst that performs surprisingly well in the esterification of FDCA to make an esterification product comprising a diester of FDCA with an alcohol, the catalyst being in either a bulk, unsupported form or in the form of a supported tin (II) catalyst, in particular, using a hygroscopic support such as a gamma alumina, a zeolite or a silica, or using a carbon support.

From another perspective, the present invention relates to a combination of such a heterogeneous tin (II) catalyst with at least one material having water-removing or -segregating capabilities (that is distinct from the embodiment of the heterogeneous tin (II) catalyst using a hygroscopic support) in either a mixture in a reactor, in a zoned arrangement having a first zone comprising the heterogeneous tin (II) catalyst and a second, downstream zone comprising the at least one material having water-removing or -segregating capabilities, or in a first reactor comprising the heterogeneous tin (II) catalyst and a second reactor downstream of the first which comprises the at least one material having water-removing or -segregating capabilities, wherein the addition of the at least one material having water-removing or -segregating capabilities contributes to a greater conversion of FDCA to an esterification product comprising a greater proportion of diesters, and especially of a 2,5-diester, of FDCA with an alcohol (versus monoesters formed from FDCA and the alcohol), as compared to a scenario wherein FDCA is reacted with the alcohol under the same conditions and in the presence of the heterogeneous tin (II) catalyst of the present invention, but in the absence of the at least one material having water-removing or -segregating capabilities.

From still another perspective, the present invention relates broadly to a method for forming one or more esters of FDCA, wherein an FDCA-containing feed is reacted with an alcohol in the presence of a tin-containing heterogeneous catalyst, such that a substantial improvement is realized in the amounts produced of the corresponding 2,5-diester of FDCA with the alcohol over what would be produced autocatalytically at the same temperature and over the same time period, while concurrently providing a 2,5-FDCA diester product whose APHA color as formed as determined under ASTM D1209 is comparable to, or at least not greatly more than, the APHA color of the same 2,5-FDCA diester product as produced autocatalytically (again at the same temperature and over the same time period), while in especially preferred embodiments providing a 2,5-FDCA diester product with an improved APHA color value without further refining or color improvement measures being necessary.

In certain embodiments of this method, the tin-containing heterogeneous catalyst used in the method is a heterogeneous tin (II) catalyst according to the present invention.

In certain embodiments of the method, the method includes using a heterogeneous tin (II) catalyst of the present invention in a combination with at least one material having water-removing or -segregating capabilities as summarized above.

In certain embodiments of the method, the FDCA-containing feed is in the form of a fully-liquid FDCA-containing feed mixture suitable for reaction with an alcohol in the presence of a tin-containing heterogeneous catalyst in a fixed bed of a fixed bed reactor, and the fully-liquid FDCA-containing feed mixture is prepared by reacting a supply of FDCA first with the alcohol but in the absence of any extrinsic esterification catalyst to provide a fully-liquid FDCA-containing feed mixture also comprising (in addition to FDCA) both monoesters and diesters of FDCA with the alcohol, and excess alcohol.

In certain embodiments, the supply of FDCA is in the form of a slurry of FDCA solids in a liquid medium that comprises the alcohol with which the FDCA is to be reacted.

In certain embodiments, the liquid medium further comprises an additional solvent for FDCA.

In certain embodiments, the additional solvent comprises a recycle portion of a mono- or diester of FDCA formed in the esterification.

In certain embodiments, the liquid medium consists essentially of a combination of the alcohol with which the FDCA is to be reacted with a recycle portion of a product diester of FDCA.

In certain other embodiments of the method employing a fully-liquid FDCA-containing feed mixture suitable for reaction with an alcohol in the presence of a tin-containing heterogeneous catalyst in a fixed bed of a fixed bed reactor, the fully-liquid FDCA-containing feed mixture is prepared by combining a supply of FDCA with one or more solvents for the FDCA and forming a solution of FDCA in the one or more solvents, and this solution is then supplied as the fully-liquid FDCA-containing feed mixture alongside the alcohol to a fixed bed reactor containing the tin-containing heterogeneous catalyst in a fixed bed, such that FDCA in the fully-liquid FDCA-containing feed mixture is reacted with the alcohol in the presence of the tin-containing heterogeneous catalyst to produce an esterification product comprising at least a 2,5-diester of FDCA with the alcohol of the prescribed low-color character and yet with the prescribed improvements in productivity compared to producing the same material autocatalytically.

In certain embodiments wherein a fully-liquid FDCA-containing feed (however produced, whether, for example, by dissolution of a supply of FDCA in one or more selected solvents, or by reacting a supply of FDCA first with the alcohol but in the absence of any extrinsic esterification catalyst to provide a fully-liquid FDCA-containing feed also comprising (in addition to FDCA) both monoesters and diesters of FDCA with the alcohol and further comprising excess alcohol) is supplied with an alcohol to a fixed bed reactor containing the tin-containing heterogeneous catalyst, and FDCA in the feed is then reacted with the alcohol in the presence of the tin-containing heterogeneous catalyst in the fixed bed reactor to produce an esterification product comprising at least a 2,5-diester of FDCA with the alcohol of the prescribed low-color character, a plurality of such fixed bed reactors are employed such that the esterification product can be continuously produced while regenerating at least one such fixed bed reactor (and “a plurality of fixed bed reactors” here will be understood as including a plurality of fixed beds or active catalytic zones within a single reactor vessel).

In certain embodiments, the fixed bed reactor or plurality of reactors wherein the esterification reaction takes place is (or are) preceded by at least one guard bed through which an FDCA-containing feed is processed prior to entry into the fixed bed reactor or plurality of reactors, and humins and any other undesirable organic impurities present in the FDCA-containing feed are sequestered therein along with dissolved Co⁺² and Mn⁺² from the remainder of the FDCA-containing feed, prior to that remainder's then being supplied to the fixed bed reactor or plurality of reactors for reacting with an alcohol and forming an esterification product comprising at least a 2,5-diester of FDCA with the alcohol. The humins and other organic impurities can be burned off to regenerate a guard bed, and the dissolved Co⁺² and Mn⁺² recovered for recycling and reuse by washing.

In certain other embodiments, the fixed bed reactor or plurality of fixed bed reactors are not preceded by a guard bed or beds, and the humins and other organic impurities together with dissolved Co⁺² and Mn⁺² are removed from the fixed bed reactor or plurality of reactors as part of the regeneration of the heterogeneous tin-containing catalyst deployed therein.

### BRIEF DESCRIPTION OF THE DRAWING

FIG. 1 is a schematic illustration of a process of the present invention in one illustrative embodiment.

### DETAILED DESCRIPTION OF CERTAIN ILLUSTRATIVE EMBODIMENTS

The disclosures of all patent and non-patent literature referenced herein are hereby incorporated in their entireties.

As used in this application, the singular forms “a”, “an” and “the” include plural references unless the context clearly indicates otherwise. The term “comprising” and its derivatives, as used herein, are similarly intended to be open ended terms that specify the presence of the stated features, elements, components, groups, integers, and/or steps, but do not exclude the presence of other unstated features, elements, components, groups, integers and/or steps. This understanding also applies to words having similar meanings, such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers, and/or steps. The term “consisting essentially of”, as used herein, is intended to specify the presence of the stated features, elements, components, groups, integers, and/or steps, as well as those that do not materially affect the basic and novel characteristic(s) of stated features, elements, components, groups, integers, and/or steps. Terms of degree such as “substantially”, “about” and “approximately” as used herein mean a reasonable amount of deviation of the modified term (beyond that degree of deviation understood by the precision (significant figures) with which a quantity is expressed) such that the end result is not significantly changed. These terms of degree should be construed as including a deviation of at least plus or minus five (5) percent from the stated value, provided this deviation would not negate the meaning of the term modified.

Where specific numerical values are used to quantify certain parameters relating to the invention without an accompanying term of degree, and where the specific numerical values are not expressly part of a numerical range, it will be understood that each such specific numerical value provided herein is to be construed as providing literal support for a broad, intermediate and narrow range of values for the parameter in question. The broad range shall be the numerical value plus and minus 60 percent of the numerical value, rounded to two significant digits. The intermediate range shall be the numerical value plus and minus 30 percent of the numerical value, rounded to two significant digits, while the narrow range shall be the numerical value plus and minus 15 percent of the numerical value again to two significant digits. Further, these broad, intermediate and narrow numerical ranges should be applied not only to the specific values, but also to the differences between these specific values. Thus, if the specification describes a first pressure of 110 psia for a first stream and a second pressure of 48 psia (a difference of 62 psia) for a second stream, the broad, intermediate and narrow ranges for the pressure difference between these two streams would be 25 to 99 psia, 43 to 81 psia, and 53 to 71 psia, respectively.

Where the present description uses numerical ranges to quantify certain parameters relating to the invention, it will be similarly understood that these ranges are to be construed as providing literal support for claim limitations that only recite the lower value of the range as well as claim limitations that only recite the upper value of the range.

Unless otherwise indicated, any definitions or embodiments described in this or in other sections are intended to be applicable to all embodiments and aspects of the subjects herein described for which they would be suitable according to the understanding of a person of ordinary skill in the art.

As indicated above, the present invention from one perspective concerns an improved process for esterifying furan-2,5-dicarboxylic acid, comprising reacting furan-2,5-dicarboxylic acid with one or more alcohols in the presence of a tin-containing heterogeneous catalyst providing a substantial improvement in the amounts of FDME which can be produced over what is produced autocatalytically at the same temperature and over the same time period, while concurrently providing an FDME product whose initial APHA color on recovery as determined under ASTM D1209 is comparable to, or at least not greatly more than, the APHA color of the FDME product that would be produced autocatalytically (again at the same temperature and over the same time period), and in especially preferred embodiments providing an FDME whose APHA color is also improved compared to the FDME realized autocatalytically.

In certain embodiments, a “substantial improvement” in process productivity will mean at least a 10 percent improvement in the amount of FDME produced per gram of FDCA supplied for reaction, preferably however providing at least a 20 percent improvement, more preferably at least a 30 percent improvement and still more preferably at least a 40 percent improvement in the amount of FDME produced per gram of FDCA supplied for reaction, as compared to that produced autocatalytically using the same one or more alcohols over the same time (the same batch time in a batchwise or semi-batch mode of operation, or the same residence time in a truly continuous reactor) at the same temperature in the same apparatus.

Likewise, in certain embodiments, an APHA color of the resultant heterogeneously-catalyzed FDME will be “not greatly more than” the APHA color of the FDME formed by autocatalyzed esterification when the APHA color is less than about 40 percent, preferably less than about 30 percent, more preferably less than about 20 percent and still more preferably less than about 10 percent more than the APHA color of the autocatalytically-formed FDME.

In the most preferred embodiments, both the process productivity and APHA color will be improved, so that the heterogeneously-catalyzed FDME produced will have an APHA color as determined by ASTM D1209 that is less than that of an FDME produced under the same conditions without an extrinsic acid as an esterification catalyst (i.e., autocatalytically) and yet at least a 10 percent improvement will be realized in the amount of FDME produced per gram of FDCA supplied for reaction.

We have found that these objectives—greater efficiency in converting FDCA to the desired FDME product, while simultaneously providing an FDME product whose color is not unacceptably increased by the effort to produce more FDME from a given quantity of FDCA—can be achieved in certain preferred embodiments using a heterogeneous tin (II) catalyst in either a bulk, unsupported form or on a support, in particular, on a hygroscopic support such as a gamma alumina, a zeolite or a silica, or on a carbon support. Surprisingly in light of the US'431 reference as well as the WO 2018/093413 publication, a heterogeneous tin (II) oxide catalyst substantially outperformed the heterogeneous tin (IV) catalysts that we evaluated. Surprisingly as well, the heterogeneous versus homogeneous character of the tin (II) catalysts was observed to have a substantial (positive) impact particularly in relation to the color of the FDME that was produced.

The heterogeneous tin (II) catalysts we have found capable of providing these desired outcomes can be a bulk, unsupported catalyst, for example, a bulk tin (II) oxide catalyst, which will typically be employed at a loading of from 0.1 to 10 percent by weight based on the weight of FDCA supplied to the reactor, in some embodiments being employed at from 0.5 to 5 percent by weight of the FDCA, and in other embodiments from 1 to 2 percent by weight of the FDCA supplied for the esterification, at an alcohol:FDCA molar ratio of typically at least 1:1 to not more than 20:1, especially from 1.5:1 to 10:1, and in certain embodiments of from 2:1 to 5:1, at a temperature that is typically from 140 deg. C to 220 deg. C, in other embodiments from 160 deg. C to 200 deg. C and in still other embodiments from 180 deg. C to 190 deg. C.

In other embodiments, the heterogeneous tin (II) catalysts can be supported, with the supports optionally being hygroscopic in nature, for example, a tin (II) oxide on a hygroscopic support selected from the aluminas, zeolitic materials and silicas, or in other embodiments being a carbon support. Typically, the supported tin (II) oxide catalysts will comprise from 0.5 to 10 percent by weight of tin (II) oxide on the support, preferably from 1 to 5 percent by weight and more preferably will comprise from 2 to 3 percent by weight of tin (II) oxide on the support.

In still other embodiments, a bulk tin (II) oxide catalyst or a heterogeneous tin (II) catalyst of the type described herein, whether on a hygroscopic or a non-hygroscopic support, can be used in combination with a water-scavenging material or materials (in the particular example of a supported tin (II) catalyst on a hygroscopic support, a suitable water-scavenging material can be the same hygroscopic support but absent the presence of the tin (II) catalytic component), for example, in admixture in a fixed bed arrangement or in a zoned arrangement, employing the water-scavenging material or materials to remove water formed in the esterification from a liquid phase esterification product mixture comprising the desired diester of FDCA, and thereby encourage complete esterification and greater production of the desired diester as compared to the monoester or other possible products.

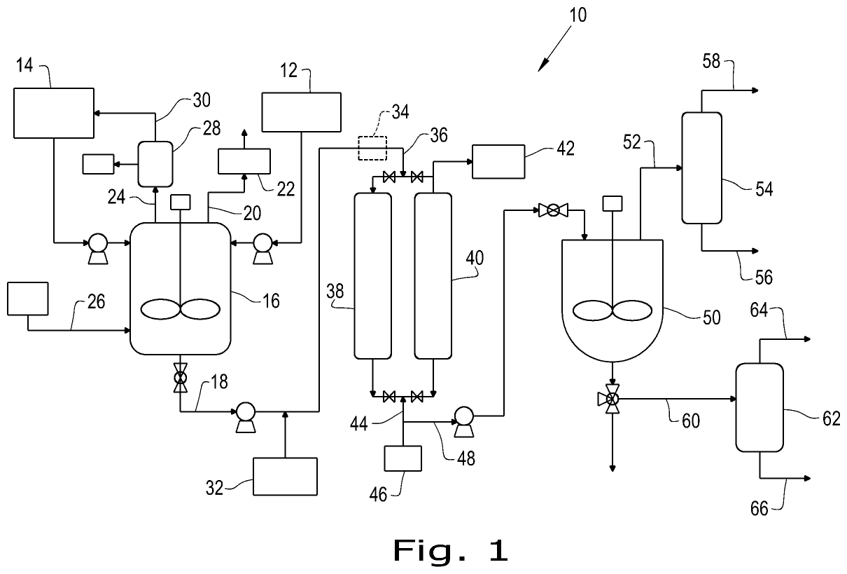

Referring now to FIG. 1, a process for making especially the desired low color, 2,5-diesters of FDCA (e.g., FDME) using a bulk tin (II) oxide catalyst or heterogeneous tin (II) catalyst as described herein and exemplified below is schematically illustrated in one possible configuration adapted for a continuous mode of operation.

A slurry of up to 30 weight percent of FDCA in methanol or a combination of methanol with one or more mono- or diesters of FDCA with methanol that have been recovered and recycled from the back end of the esterification process 10 (such as 2,5-furandicarboxylic acid, dimethyl ester (FDME)) from a source 12 of such an FDCA-containing feed is continuously supplied and combined with methanol from a source 14 in a continuous stirred tank reactor or other suitable reactor vessel 16, wherein the FDCA is reacted with the methanol at an elevated temperature and over a time in the absence of any extrinsic esterification catalyst (i.e., the reaction is autocatalyzed) to a sufficient extent that a fully-liquid FDCA-containing feed mixture 18 is obtained comprising FDCA, monomethyl and dimethyl esters of FDCA and excess methanol.

Typically, the autocatalyzed esterification in vessel 16 is carried out over a period of time ranging from about 60 minutes to about 180 minutes at a temperature of from about 160 deg. C to about 200 deg. C, at an overall molar ratio of from about 10:1 to about 5:1 of methanol:FDCA. A residence time of thirty minutes at 200 degrees Celsius and a methanol:FDCA molar ratio of 10:1 to 5:1 is expected to enable more than 99 percent of the FDCA to be converted to a fully liquid FDCA-containing feed mixture 18, for example, comprising some unconverted FDCA, a combination of its dimethyl and monomethyl esters in about a 3:1 ratio and methanol.

Volatile dimethyl ether 20 that will be formed by the acid-catalyzed dehydration of methanol in the reactor 16 is vented overhead and removed by a scrubber 22, while water is desirably continuously removed overhead in a stream 24 comprising methanol and water with the assistance of an inert nitrogen sweep gas 26 and by means of a partial condenser 28 that separates the water out of the process 10 and provides a recycle portion 30 of methanol.

Fully-liquid FDCA-containing feed mixture 18 is combined with additional methanol as needed from a methanol source or supply 32—with methanol:FDCA molar feed ratios for use with a bulk tin (II) oxide catalyst or a heterogeneous tin (II) catalyst of the present invention contemplated as ranging from 10:1 to 1:1, in certain embodiments from 7:1 to 2:1, in other embodiments from 3:1 to 6:1 and in still other embodiments from 4:1 to 5:1 and passed into a guard bed zone 34 (optionally omitted in certain embodiments of a process 10) comprised of at least a plurality of guard beds in parallel array whereby one or more such guard beds are on line and in use while one or more other such beds are being regenerated offline, with each employing an inexpensive or easily regenerable material or materials on or in which any humins and other undesired organic impurities may be captured from the feed mixture 18, together with any Co⁺² and Mn⁺² carried through from a prior Mid Century-type oxidation from which the FDCA has been generated. Materials expected to be suitable for use in the guard beds for these purposes would include molecular sieves, aluminas, zeolitic materials, silicas, carbons, zirconias and titanias.

The FDCA-containing feed 36 is then in the illustrated non-limiting embodiment supplied to a fixed bed reactor 38 (in parallel with a second, offline fixed bed reactor 40 of the same character) comprising a bulk tin (II) oxide catalyst or a heterogeneous tin (II) catalyst of the present invention in a fixed bed and preferably further comprising a water-scavenging material or materials (in the particular example of a supported tin (II) catalyst on a hygroscopic support, a suitable water-scavenging material can be the same hygroscopic support but absent the presence of the tin (II) catalytic component), for example, in admixture with the bulk tin (II) oxide catalyst or a heterogeneous tin (II) catalyst of the present invention or in a zoned arrangement with the bulk tin (II) oxide catalyst or a heterogeneous tin (II) catalyst of the present invention within the fixed bed reactors 38 and 40. A dimethyl ether scrubber 42 is again employed to receive and remove volatile DME generated by the dehydration of methanol in operation of a reactor 38 or 40, facilitated by a nitrogen drying and sweep gas 44 from a source 46 of the same.

The heterogeneous tin (II) catalyst can again be a bulk, unsupported catalyst, for example, a bulk tin (II) oxide catalyst, which will typically be employed at a loading of from 0.1 to 10 percent by weight based on the weight of FDCA supplied to the reactor, in some embodiments being employed at from 0.5 to 7 percent by weight of the FDCA, and in other embodiments from 1 to 5 percent by weight of the FDCA supplied for the esterification.

In other embodiments, the heterogeneous tin (II) catalyst can be supported, with the supports optionally being hygroscopic in nature, for example, a tin (II) oxide on a hygroscopic support selected from the aluminas, zeolitic materials and silicas, or in other embodiments being a carbon support. Typically, the supported tin (II) oxide catalysts will comprise from 0.1 to 10 percent by weight of tin (II) oxide on the support, preferably from 0.5 to 5 percent by weight and more preferably will comprise from 2 to 4 percent by weight of tin (II) oxide on the support.

In the context of a fixed catalyst bed of a continuous process, those of skill in the art will appreciate that bulk, unsupported tin (II) oxide catalysts that are in a smaller particulate form may be aggregated or agglomerated into larger particles with the mechanical properties desired for use in that context, for example, through the use of an inert binder, and in particular embodiments may desirably be formed into an extrudate that is particularly adapted for use in a fixed bed process as illustrated; it will be understood, consequently, that “heterogeneous bulk, unsupported tin (II) catalysts” and the like as used herein shall include compositions and resulting aggregated, agglomerated and extruded or otherwise formed constructs in which a bulk tin (II) oxide particulate and an inert binder have been combined to better adapt the bulk tin (II) oxide particulate for use in a particular process configuration, for example, a fixed catalyst bed. Similarly, a “supported tin (II) oxide catalyst” as used herein shall be understood as encompassing agglomerates, aggregates, extrudates and other formed constructs wherein especially the hygroscopic materials contemplated as supports herein have been combined with an inert binder and optionally with other support materials to provide an agglomerate, aggregate, extrudate or other formed construct, for example.

In reactors 38 and 40, as demonstrated by the examples following, the FDCA in the feed 36 can be virtually quantitatively converted to its monoester acid and diester derivatives, being at least about 60 percent converted, more preferably at least about 80 percent converted, still more preferably being at least about 90 percent converted and even more preferably being at least 99 percent converted at reasonable temperatures of about 200 degrees Celsius or less and in a reasonable average residence time of about 180 minutes or less.

Switching to an offline reactor in parallel will typically be undertaken on water breakthrough from the reactor (or reactors) then online, with regeneration of the water-removing capacity of the materials in the reactor then online following.

Product mixture 48 is then conveyed to product tank 50 maintained under reduced pressure, with a vapor phase fraction 52 comprising methanol, water, 2-methylfuroate and some FDME being drawn overhead from the product tank 50 to be subsequently distilled in a lights column 54 to provide an FDME bottoms stream 56 and an overhead stream 58 comprised of preferably everything else contained in vapor phase fraction 52, and with a liquid phase fraction 60 comprised of FDME and any residual heavier, higher molecular weight material being passed from product tank 50 to heavies distillation column 62 for then providing a condensable vapor phase FDME product stream 64 and a heavies residue stream 66 comprising preferably everything else (e.g., residual humins) contained in the liquid phase fraction 60.

The following, non-limiting examples of features and combinations of features addressed above collectively further illustrate the present invention:

### EXAMPLES

### Examples 1 and 2 with Comparative Example 1

Pellets of 1% stannous (tin (II)) oxide supported on an extruded activated carbon support (Norit® activated carbon, Boston, MA) were first prepared following the following prescribed protocol:

0.508 grams of tin oxalate were dissolved in approximately 15 ml of 2.8 molar hydrochloric acid. The solution was sprayed onto the carbon while rotating using a Sonaer atomizer nozzle (Sonaer, Inc., Farmingdale, NY) and syringe pump. The spraying device was rinsed with 5 ml of water to spray any residual metal solution and bring the total spray volume to 20 ml. The carbon was allowed to spin under air flow for approximately 30 minutes. The material was then placed in a tube furnace for drying and calcination. The furnace was ramped to 100° C. under nitrogen and held for 20 minutes. It was then ramped to 350° C. and held for 100 minutes. The catalyst was then allowed to cool overnight under flowing nitrogen.

A 75 cc Hasteloy autoclave equipped with a glass enclosed magnetic stirrer was then charged for each of two runs with 6 grams of FDCA and 24 grams of methanol. In a first example, 1 gram of the carbon-supported tin (II) oxide catalyst (providing 0.010 grams of stannous oxide, 0.17 weight percent of the FDCA fed) was then added, and in a second instance, 2.5 grams of catalyst was added (affording 0.025 grams of stannous oxide, 0.42 percent by weight of the FDCA fed). The autoclave was then sealed with a pressure head containing a thermocouple and pressure transducer, and then placed in a heating well. While stirring at 1000 rpm, the autoclave was in each run heated to 200 degrees Celsius over the course of 1 hour (employing a 35-minute heat up time). After the hour, the vessel was flash cooled in an ice bath and the contents were removed when the temperature had reached 15 degrees Celsius.

The residual wetcake generated from each run was dissolved in acetonitrile, and the heterogeneous carbon-supported stannous oxide catalyst vacuum filtered through a Celite pad and recovered. Each filtrate was then dried under reduced pressure, affording in both instances an off-white powder.

Analysis by UPLC-PDA for the 1 gram of catalyst example indicated that 98.4% of the FDCA had converted, producing an esterification product mixture containing 22.9% by weight of FDMME (monomethyl ester), 75.5% by weight of FDME and the remainder (1.6% by weight) of other products including the unconverted FDCA.

Analysis by UPLC-PDA for the 2.5 grams of catalyst example indicated that 98.8% of the FDCA had converted, producing an esterification product mixture containing 12.6% by weight of FDMME (monomethyl ester), 86.2% by weight of FDME and the remainder (1.2% by weight) of other products including the unconverted FDCA.

For comparison, a run employing only the same activated carbon support but without the presence of any stannous oxide thereon under the same conditions and employing the same reaction protocol showed a fairly substantial conversion of the FDCA (96.4% converted) but produced much more of the monoester (33.9% by weight of FDMME) and much less of the desired diester (62.5% by weight FDME) alongside the remainder including the unconverted FDCA (3.6% by weight in total).

### Example 3

For this example, pellets of 1% stannous (tin (II)) oxide supported on an extruded activated carbon support (Norit® activated carbon, Boston, MA) were again prepared, but with a longer drying time, following the following prescribed protocol:

0.405 grams of tin oxalate were dissolved in approximately 16 ml of 3 molar hydrochloric acid. The solution was sprayed onto the carbon while rotating using a Sonaer atomizer nozzle (Sonaer, Inc., Farmingdale, NY) and syringe pump. The spraying device was rinsed with 5 ml of water to spray any residual metal solution and bring the total spray volume to 20 ml. The carbon was allowed to spin under air flow for approximately 30 minutes. The material was then placed in a tube furnace for drying and calcination. The furnace was ramped to 100° C. under nitrogen and held for 120 minutes rather than 20 as in Examples 1 and 2. It was then ramped to 350° C. and held for an additional 120 minutes. The catalyst was then allowed to cool overnight under flowing nitrogen.

Using the same equipment and protocol as used for the 2.5 wt. percent catalyst run (Example 2) provided—as per UPLC-PDA analysis—0.98% by weight of unconverted FDCA, 13.5 weight percent of FDMME, 85.5 weight percent of FDME and 0.02% by weight of furoic acid.

### Example 4

For this example, a 5% loading stannous oxide catalyst was prepared and evaluated using the same extruded activated carbon and the same experimental apparatus and protocol as in prior examples. The 5% catalyst was prepared by dissolving 2.083 grams of tin oxalate in approximately 15 ml of 3 molar hydrochloric acid. 6 ml of 12 molar hydrochloric acid was then added to fully dissolve the tin oxide. The solution was sprayed onto the carbon while rotating using the same Sonaer atomizer nozzle and syringe pump. The spraying device was rinsed with water to spray any residual metal solution. The carbon was allowed to spin under air flow for approximately 30 minutes. The material was then placed in a tube furnace for drying and calcination. The furnace was ramped to 100° C. under nitrogen and held for 120 minutes. It was then ramped to 350° C. and held for 120 minutes. The catalyst was then allowed to cool overnight under flowing nitrogen.

One gram of the catalyst thus prepared (providing 0.050 grams of stannous oxide, or 0.83 percent by weight of the FDCA fed) was evaluated in the esterification of FDCA as in previous examples. The esterification product mixture was determined to include 9.1% by weight of FDMME, about 90.2% by weight of FDME, 0.02% by weight of furoic acid, a trace amount (about 30 ppm) of 2-formyl-furan-5-carboxylic acid (FFCA) and the balance of unconverted FDCA.

### Examples 5-8

For these examples, a commercial grade bulk, unsupported tin (II) oxide in the form of a black powder (acquired from Keeling & Walker Ltd., Stoke-On-Trent, United Kingdom) was evaluated in several loadings relative to the amount of FDCA supplied to be esterified with methanol.

A 75 cc Parr autoclave equipped with a glass enclosed magnetic stir bar was charged with 6 g of FDCA (20 wt. %), the indicated loading of the bulk tin (II) oxide relative to the 6 grams of FDCA and with 24 g of methanol. The vessel was sealed then heated in a block to 200° C. for 1 hour with magnetic agitation of 875 rpm (including a 30-minute heat up to get to the 200 degrees temperature). After this time, the vessel was flash cooled in an ice bath, and once reaching 25° C., the contents weighed and removed. The residual paste was dissolved entirely in tetrahydrofuran and then dried under reduced pressure. Compositional analysis of the dried esterification product was then performed on a UPLC with UV detection, while colorimetry (APHA by ASTM D1209) was conducted with a solution of 6 wt. % of the dried product mixture in equal parts by volume of isopropanol/acetonitrile. The test results are displayed in Table 1, relative to an autocatalyzed run carried out identically but with no extrinsic esterification catalyst used:

TABLE 1

| | FDCA | | | | |
|---|---|---|---|---|---|
| Catalyst Loading | Conversion | FDCA | FDMME | FDME | |
| (Wt. Pct.) | (%) | (wt. %) | (wt. %) | (wt. %) | APHA |
| 0.1 | 97.520 | 2.480 | 30.900 | 67.667 | 20 |
| 0.5 | 98.845 | 1.155 | 22.409 | 76.339 | 21 |
| 1.0 | 99.805 | 0.195 | 8.376 | 91.275 | 25 |
| 5.0 | 99.678 | 0.322 | 8.708 | 90.714 | 32 |
| None - Auto | 95.626 | 4.374 | 33.451 | 62.690 | 23 |

### Comparative Examples 2 and 3

Two samples of bulk tin (IV) oxides obtained from the same supplier (Keeling & Walker Ltd.) were evaluated in the same manner at a 5% loading to the FDCA supplied to be esterified. These were in the form of stannic or metastannic acids, hydrated forms of tin (IV) oxide, with comparatively higher surface areas ranging upwards of 35 square meters per gram (BET surface area). The results of testing on the products produced using these materials are shown in Table 2, for comparison:

TABLE 2

| | Loading | FDCA | FDMME | FDME |
|---|---|---|---|---|
| Sample | (Wt. Pct) | (wt. %) | (wt. %) | (wt. %) |
| A | 5 | 1.940 | 26.852 | 71.422 |
| B | 5 | 2.664 | 26.868 | 70.782 |

### Examples 9 and 10

Pellets of 1% stannous (tin (II)) oxide supported on two different gamma aluminas, one characterized by a BET surface area of 101 square meters per gram (corresponding to Catalyst A in Table 3 below) and the second by a BET surface area of 161 square meters per gram (corresponding to Catalyst B in Table 3), were first prepared following the following prescribed protocol:

0.508 grams of tin oxalate were dissolved in approximately 15 ml of 2.8 molar hydrochloric acid. The solution was sprayed onto the alumina in question while rotating using a Sonaer atomizer nozzle (Sonaer, Inc., Farmingdale, NY) and syringe pump. The spraying device was rinsed with 5 ml of water to spray any residual metal solution and bring the total spray volume to 20 ml. The wetted alumina was allowed to spin under air flow for approximately 30 minutes. The material was then placed in a tube furnace for drying and calcination. The furnace was ramped to 100° C. under nitrogen and held for 20 minutes. It was then ramped to 350° C. and held for 100 minutes. The catalyst was then allowed to cool overnight under flowing nitrogen.

A 75 cc Hasteloy autoclave equipped with a glass enclosed magnetic stirrer was then charged for each of two runs with 6 grams of FDCA and 24 grams of methanol. In each case, 2.5 grams of the respective alumina-supported tin (II) oxide catalyst (providing 0.025 grams of stannous oxide, 0.42 weight percent of the FDCA fed) was then added. The autoclave was then sealed with a pressure head containing a thermocouple and pressure transducer, and then placed in a heating well. While stirring at 1000 rpm, the autoclave was in each run heated to 200 degrees Celsius over the course of 1 hour (employing a 35-minute heat up time). After the hour, the vessel was flash cooled in an ice bath and the contents were removed when the temperature had reached 15 degrees Celsius.

The residual wetcake generated from each run was dissolved in acetonitrile, and the heterogeneous carbon-supported stannous oxide catalyst vacuum filtered through a Celite pad and recovered. Each filtrate was then dried under reduced pressure, affording in both instances an off-white powder.

Analysis by UPLC-PDA gave the following results in Table 3:

TABLE 3

| | FDCA | FDMME | FDME |
|---|---|---|---|
| Catalyst | (Wt. %) | (Wt. %) | (Wt. %) |
| A | 1.0 | 15.2 | 83.4 |
| B | 1.0 | 14.1 | 85.0 |

What is claimed is:

1. A process for esterifying furan-2,5-dicarboxylic acid, comprising reacting an alcohol with furan-2,5-dicarboxylic acid in the presence of a tin-containing heterogeneous catalyst in a reactor vessel to produce at least 10 percent more of the diester of the alcohol with the furan-2,5-dicarboxylic acid per gram of furan-2,5-dicarboxylic acid supplied for reaction with the alcohol, as compared to that amount of diester that would be produced per gram of furan-2,5-dicarboxylic acid supplied for reaction with the alcohol from the same amounts of the same alcohol under the same reaction conditions except in the absence of any extrinsic catalyst for the esterification, and wherein the diester produced nevertheless has an APHA color on recovery that is less than about 40 percent greater than the APHA color of the diester that would be produced by reacting the furan-2,5-dicarboxylic acid with the same alcohol and under the same reaction conditions except in the absence of any extrinsic catalyst for the esterification.

2. The process of claim 1, wherein the APHA color of the diester is less than about 30 percent greater than the APHA color of the diester produced in the absence of any extrinsic catalyst.

3. The process of claim 2, wherein the APHA color of the diester is less than about 20 percent greater.

4. The process of claim 3, wherein the APHA color of the diester is less than about 10 percent greater.

5. The process of any of claims 1-4, wherein the tin-containing heterogeneous catalyst comprises one or more metals inclusive at least of tin on a carbon support.

6. The process of any of claims 1-5, further comprising removing water as the reaction proceeds.

7. The process of claim 6, wherein water is removed as the reaction proceeds by providing a quantity of an inert, hygroscopic material in an intimate mixture with the tin-containing heterogeneous catalyst in the reactor vessel.

8. The process of claim 7, wherein the inert, hygroscopic material is one or more of the aluminas, silicas and zeolites.

9. The process of claims 1-8, wherein the tin-containing heterogeneous catalyst comprises one or more metals inclusive at least of tin on a hygroscopic support.

10. The process of claim 9, wherein the hygroscopic support is a gamma alumina, a zeolite or silica.

11. The process of claim 10, further comprising removing water as the reaction proceeds by providing a quantity of an inert, hygroscopic material in an intimate mixture with the tin-containing heterogeneous catalyst in the reactor vessel.

12. The process of claim 11, wherein the inert, hygroscopic material is one or more of the aluminas, silicas and zeolites.

13. The process of any of claims 1-12, wherein the tin-containing heterogeneous catalyst is a tin (II) catalyst.

14. A process for esterifying furan-2,5-dicarboxylic acid, comprising reacting an alcohol with furan-2,5-dicarboxylic acid in the presence of a heterogeneous tin (II) catalyst in a bulk, unsupported form or in the form of a supported catalyst comprising one or metals inclusive of tin (II) on a support.

15. The process of claim 14, wherein the heterogeneous tin (II) catalyst is an unsupported tin (II) oxide.

16. The process of claim 14, wherein the catalyst is tin (II) oxide on a support.

17. The process of claim 16, wherein the support is a carbon support.

18. The process of claim 16, wherein the support is a hygroscopic support selected from the gamma aluminas, the zeolites and silica.

19. The process of any of claims 14-18, further comprising removing water as the reaction proceeds by providing a quantity of an inert, hygroscopic material in an intimate mixture with the tin-containing heterogeneous catalyst in the reactor vessel.

20. The process of claim 19, wherein the inert, hygroscopic material is one or more of the aluminas, silicas and zeolites.

21. A process for esterifying furan-2,5-dicarboxylic acid, comprising:
reacting an alcohol with furan-2,5-dicarboxylic acid in a first, partial esterification step without the use of any extrinsic catalyst to form a first esterification product including both monoester and diester derivatives as well as unconverted furan-2,5-dicarboxylic acid; and
contacting some or all of the first esterification product with a second quantity of the alcohol in the presence of a heterogeneous tin (II) catalyst in a bulk, unsupported form or in the form of a supported catalyst comprising one or metals inclusive of tin (II) on a support to form more of the diester derivative of furan-2,5-dicarboxylic acid in a second, polishing esterification step.

22. The process of claim 21, wherein the heterogeneous tin (II) catalyst is an unsupported tin (II) oxide.

23. The process of claim 21, wherein the catalyst is tin (II) oxide on a support.

24. The process of claim 23, wherein the support is a carbon support.

25. The process of claim 23, wherein the support is a hygroscopic support selected from the gamma aluminas, the zeolites or silica.

26. The process of any of claims 21-25, further comprising removing water as the reaction proceeds by providing a quantity of an inert, hygroscopic material in an intimate mixture with the tin-containing heterogeneous catalyst in the reactor vessel.

27. The process of claim 26, wherein the inert, hygroscopic material is one or more of the aluminas, silicas and zeolites.
